(19) United States
(12) Patent Application Publication
(10) Pub. No.: US 2025/0134671 A1
(43) Pub. Date: May 1, 2025

(54) Glenoid Baseplate

(71) Applicant: CORENTEC CO., LTD, Seoul (KR)

(72) Inventors: Jung-Sung Kim, Seoul (KR); Jae-Won Kim, Seoul (KR); Sung-Wook Jung, Seoul (KR); Sang-Kil Lee, Hanam-si, Gyeonggi-do (KR); Yeon-Beom Heo, Seoul (KR)

(21) Appl. No.: 18/706,309

(22) PCT Filed: Oct. 17, 2022
(86) PCT No.: PCT/KR2022/015688

§ 371 Date: Apr. 30, 2024

(30) Foreign Application Priority Data
Nov. 3, 2021 (KR) 10-2021-0149488

(51) Int. Cl. A61F 2/40; A61F 2/30

(52) U.S. Cl. CPC A61F 2/4081; A61F 2/30942; A61F 2002/30736

(57) ABSTRACT

A glenoid baseplate and, more specifically, to a glenoid baseplate of an artificial shoulder joint, includes: a base which is mounted on the glenoid fossa of the scapula and has a first surface; an augment disposed on the first surface; and a stem extended from the first surface while having a center axis, wherein one surface of the augment forms a contact surface with the first surface, at least a portion of the contact surface forms a predetermined angle, that is not a right angle, with respect to the center axis of the stem, and the augment includes a first wedge having a second surface extended from a first boundary and a second wedge having a third surface bent and extended from the second surface, and thus the present invention compensates for bone defects, minimizes bone cutting, and has a simple shape so as to facilitate manufacturing.

### TECHNICAL FIELD

The present disclosure relates to a glenoid baseplate. More particularly, the present disclosure relates to a glenoid baseplate of an artificial shoulder joint. The glenoid baseplate comprises a base having a first surface settled on the scapular glenoid, an augment that is disposed on the first surface, and a stem that is extended with a central axis at the first surface.

The first surface and one surface of the augment form a contact surface, and at least a portion of the contact surface is formed to form a predetermined angle which is not perpendicular to the central axis of the stem, and the augment includes a first wedge having a second surface extending from a first boundary and a second wedge having a third surface bending and extending from the second surface, thereby compensating bone defect, minimizing bone cutting, and having a simple shape which allows easy manufacturability.

### BACKGROUND ART

An artificial shoulder joint is a type of artificial prosthesis that replaces the shoulder joint of a human when the shoulder joint is not functioning properly. The artificial shoulder joint includes a stem that is implanted into the humerus, a glenoid baseplate coupled to the shoulder blade (scapula), and an artificial bone head and an insert that are realizing a rotational movement between the stem and the glenoid baseplate. The artificial bone head may be coupled to a stem like the shoulder joint of a human, or may be coupled to a glenoid baseplate. Accordingly, the insert is respectively coupled to the side of the glenoid baseplate and the stem.

Referring to FIG. 1, scapula 91 has a substantially inverted triangle structure, and includes subscapular fossa 913 that is a front surface of a body facing an anterior direction, infraspinous fossa (not illustrated) that is a rear surface of the body facing a posterior direction, coracoid 917 that protrudes from an upper side toward the anterior direction, acromion 919 that protrudes from an upper side toward the posterior direction, and glenoid 911 which faces a lateral direction from between the coracoid 917 and the acromion 919 and which is in contact with the caput humeri so as to realize a joint movement.

Generally, when a total joint arthroplasty of an artificial shoulder joint is performed, a glenoid baseplate is coupled to the glenoid 911 so that the glenoid baseplate replaces a function of the glenoid 911. At this time, a fixing means such as a screw is used to securely couple the glenoid baseplate to the glenoid 911.

### Patent Document

U.S. Pat. No. 9,132,016 (registered on Sep. 15, 2015) “Implantable shoulder prostheses”

The invention disclosed in the patent document described above discloses a glenoid baseplate used in an artificial shoulder joint.

However, the conventional baseplate such as the prior art featured in FIG. 2, forming a flat lower surface 81 in the direction from which the stem having hollow extends. Such a conventional baseplate has a disadvantage of not being able to be settled or inserted stably when bone loss occurs in glenoid. When a conventional baseplate that cannot compensate for bone loss is inserted and fixed in the scapular glenoid 911 which bone loss have occurred, the portion R1 where the bone loss has not occurred must be cut for settling the glenoid baseplate, as shown in FIG. 3. On the other hand, when the baseplate is settled on the glenoid without bone cutting, there is a problem that the baseplate cannot be settled on a scapula 91 or glenoid 911 in the portion R2 where the bone loss has occurred, as shown in FIG. 4.

### DISCLOSURE

### Technical Problem

The present disclosure has been conceived in order to solve the problems described above.

An objective of the present disclosure is to provide a glenoid baseplate capable of compensating bone loss, the glenoid baseplate comprising a base that has a first surface and is disposed on the scapular glenoid, an augment disposed on the first surface, and a stem having a central axis and extending from the first surface. The first surface and one surface of the augment forms a contact surface, wherein at least a portion of the contact surface forms a predetermined angle with the central axis of the stem that is not perpendicular.

Another objective of the present disclosure is to provide a glenoid baseplate capable of stably transmitting force near the center where stress is concentrated, by forming the augment to be spaced apart from the central axis, and forming one surface of the augment to be inclined while having a first angle with the first surface in one direction from a point spaced a first length apart from the central axis.

In addition, another objective of the present disclosure is to provide a glenoid baseplate capable of stable fixation by reducing concentrated stress and having a small displacement when an external force is applied, wherein the augment comprises a first wedge having a second surface extending from a first boundary and a second wedge having a third surface bending and extending from the second surface.

In addition, yet another objective of the present disclosure is to provide a glenoid baseplate in which bone cutting is minimized by having a second angle of the third surface and the first surface greater than a first angle of the second surface and the first surface.

In addition, yet another objective of the present disclosure is to provide a glenoid baseplate, which is easily manufactured in a simple shape by forming a second boundary formed by the second surface and third surface as a straight line that is spaced a second length apart from a first boundary.

In addition, yet another objective of the present disclosure is to provide a glenoid baseplate that can ensure stability with minimal processing by forming the first length to be less than the distance between the central axis and outer surface of the stem, and the distance between the central axis and the second boundary to be less than the distance between the central axis and the outer surface of the stem.

In addition, yet another objective of the present disclosure is to provide a glenoid baseplate comprising a porous layer having a predetermined thickness on one side of the base and the stem, which is formed to coat the surface of the base and the stem and has plural pores therein, wherein the porous layer has a complementary shape to the base and the stem so that bone growth can be promoted between pores.

In addition, yet another objective of the present disclosure is to provide the base comprising a central fixing hole formed to vertically pass through the base and stem, a peripheral fixing hole formed around the central fixing hole, and a flange projecting from a surface of the base along the edge of the peripheral fixing hole, wherein one end of the stem and an edge of fixing hole vertically penetrating the base are exposed without being covered by the porous layer, so that the glenoid baseplate may prevent the porous layer from being broken or dislodged by fixing means.

In addition, yet another objective of the present disclosure is to provide a glenoid baseplate maximizing the space available for bone growth by forming an augment as a porous structure having plural pores therein.

In addition, yet another objective of the present disclosure is to provide a glenoid baseplate reducing the weight of the stem while providing enough strength to withstand athletic loads on the shoulder joint, wherein the stem comprises a reinforcement member protruding and extending along outer surface, and the reinforcement member comprises a rib protruding with a predetermined width from one end of the base to the other end of the base, and a rim protruding in a ring shape with a predetermined width from at least one end of the stem.

In addition, yet another objective of the present disclosure is to provide a glenoid baseplate having different thickness between the relatively strongly stressed portion and weakly stressed portion to express appropriate strength and maximize weight lightening by forming the recessed part formed in at least one surface of the base is recessed at at least a predetermined distance from the edge of at least one of central fixing hole, the peripheral fixing hole, and the edge of the base.

In addition, yet another objective of the present disclosure is to provide a glenoid baseplate, in which a base, an augment and a porous layer are integrally formed so as not to be separated from each other so that a member for combining the components can be omitted.

### Technical Solution

The present disclosure may be implemented by one or more embodiments having some or all of the following configurations, to achieve one or more of the above-described objectives.

According to an embodiment of the present disclosure, the present disclosure comprises a base configured to be settled on a scapular glenoid and having a first surface, an augment disposed on the first surface, and a stem extending from the first surface with a central axis, wherein the first surface and one surface of the augment make a contact surface, while at least a part of the contact surface makes a predetermined angle which is not perpendicular to the central axis of the stem.

According to another embodiment of the present disclosure, the augment is formed to be spaced apart from the central axis, and one surface of the augment is formed to be inclined while having a first angle with the first surface in one direction from a point spaced a first length apart from the central axis.

According to yet another embodiment of the present disclosure, a first boundary between the augment and the first surface is formed as a straight line that is space a first length apart from a straight line passing through the central axis on the first surface.

According to yet another embodiment of the present disclosure, the augment comprises a first wedge having a second surface extending from the first boundary, and a second wedge having a third surface bending and extending from the second surface.

According to yet another embodiment of the present disclosure, a second angle formed by the third surface and the first surface is greater than the first angle formed by the second surface and the first surface.

According to yet another embodiment of the present disclosure, a second boundary formed by the second surface and the third surface is formed as a straight line spaced a second length apart from a first boundary.

According to yet another embodiment of the present disclosure, the first length is less than the distance between the central axis and an outer surface of the stem.

According to yet another embodiment of the present disclosure, the distance between the central axis and a second boundary is less than the distance between the central axis and the outer surface of the stem.

According to yet another embodiment of the present disclosure, the glenoid baseplate further comprises a porous layer having a predetermined thickness on one side of the base and the stem, which is formed to coat surfaces of the base and the stem, and has plural pores therein, wherein the porous layer has a complementary shape to the base and the stem.

According to yet another embodiment of the present disclosure, the base comprises a central fixing hole formed to vertically pass through the base and the stem, a peripheral fixing hole formed around the central fixing hole, and a flange protruding from a surface of the base along the edge of the peripheral fixing hole, wherein one end of the stem and an edge of fixing hole penetrating the base are exposed without being covered by the porous layer.

According to yet another embodiment of the present disclosure, an augment is formed as a porous structure having plural pores therein.

### Advantageous Effects

The present disclosure is capable of obtaining the following effects through combinations of the above-described embodiments with configurations to be described below and use relationships thereof.

According to the present disclosure, the glenoid baseplate can compensate bone loss by including a base having a first surface and disposed on the scapular glenoid, an augment disposed on the first surface, and a stem having a central axis and extending from the first surface, wherein the first surface and a surface of the augment forms a contact surface, wherein at least a portion of the contact surface forms a predetermined angle with the central axis of the stem that is not perpendicular.

According to the present disclosure, the augment is formed to be space apart from the central axis, and a surface of the augment is inclined at a first angle to the first surface in one direction from a point spaced a first length apart from the central axis, thereby providing a glenoid baseplate capable of stably transmitting force near a center where stress is concentrated.

According to the present disclosure, the augment comprises a first wedge having a second surface extending from a first boundary and a second wedge having a third surface bending and extending from the second surface, so that concentrated stress is reduced and displacement is small when an external force is applied, thereby achieving stable fixation.

According to the present disclosure, a second angle formed by the third surface and the first surface is greater than a first angle formed by the second surface and the first surface, thereby minimizing bone cutting.

According to the present disclosure, a second boundary formed by the second surface and the third surface is a straight line spaced a second length apart from a first boundary, providing a glenoid baseplate that is easily manufactured in a simple shape.

According to the present disclosure, the first length is less than a distance between a central axis and an outer surface of the stem, and a distance between central axis and a second boundary is less than a distance between central axis and an outer surface of the stem, so that stability can be secured with minimal processing.

According to the present disclosure, the glenoid baseplate comprises a porous layer having a predetermined thickness on one side of the base and the stem, forming to coat surfaces of the base and the stem, and having plural pores therein, wherein the porous layer has a complementary shape to the base and the stem to promote bone growth between the pores.

According to the present disclosure, the base comprises a central fixing hole formed to vertically pass through the base and stem, a peripheral fixing hole formed around the central fixing hole, and a flange projecting from a surface of the base along the edge of the peripheral fixing hole, wherein one end of the stem and an edge of fixing hole vertically penetrating the base are exposed without being covered by the porous layer, so that the porous layer is prevented from being broken or dislodged by fixing means.

According to the present disclosure, an augment is formed as a porous structure having plural pores therein to maximize the space available for bone growth.

According to the present disclosure, the stem comprises a reinforcement member protruding along outer surface, where the reinforcement member comprises a rib protruding with a predetermined width from one end of the base to the other end of the base, and a rim protruding in a ring shape with a predetermined with from at least one end of the stem, thereby reducing the weight of the stem while providing enough strength to withstand athletic loads on the shoulder joint.

According to the present disclosure, a recessed part recessed at a predetermined depth in at least one surface of the base is recessed at a predetermined distance from the edge of at least one of the central fixing hole, the peripheral fixing hole, and the edge of the base, so that the thickness of the relatively strongly stressed portion and weakly stressed portion are made differently, thereby expressing appropriate strength and maximizing weight lightening.

According to the present disclosure, a base, an augment and a porous layer are integrally formed so as not to be separated from each other so that a member for combining the components can be omitted, thereby providing a glenoid baseplate that is stable easily combined.

### DESCRIPTION OF DRAWINGS

FIG. 1 is a perspective view illustrating a glenoid baseplate being inserted into scapula.

FIG. 2 is a view illustrating a conventional glenoid baseplate according to prior art.

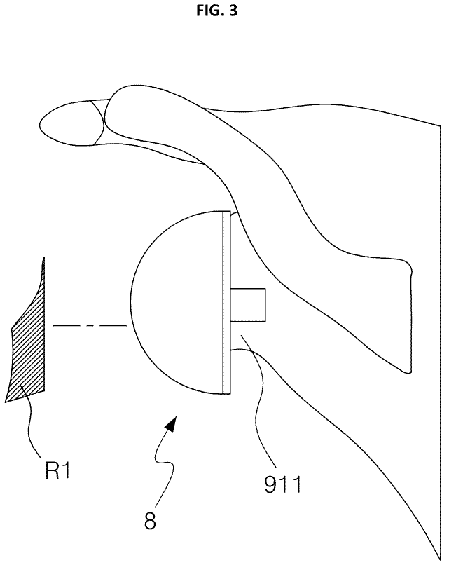

FIG. 3 and FIG. 4 are views illustrating the settling of a conventional glenoid baseplate according to the prior art on a glenoid with bone loss.

FIG. 5 is a perspective view illustrating a glenoid baseplate according to an embodiment of the present disclosure.

FIG. 6 is a plan view illustrating the glenoid baseplate according to an embodiment of the present disclosure when viewed from a first side.

FIG. 7 is an elevation view illustrating the glenoid baseplate according to an embodiment of the present disclosure when viewed from a first side.

FIG. 8 is an exploded perspective view illustrating the glenoid baseplate including a porous layer according to another embodiment of the present disclosure.

FIG. 9 and FIG. 10 are glenoid baseplates with an augment formed thereon according to other embodiments of the present disclosure.

FIG. 11 is a glenoid baseplate with a stem 15 formed thereon according to other embodiments of the present disclosure.

FIG. 12 is an exploded perspective view of a glenoid baseplate according to another embodiment of the present disclosure.

FIG. 13 is a perspective view illustrating a porous layer 3 according to an embodiment of the present disclosure.

FIG. 14 is stress formed when an external force acts on a glenoid baseplate with a second surface and a third surface according to an embodiment of the present disclosure.

FIG. 15 is stress formed when an external force acts on a glenoid baseplate with only one surface of slope.

FIG. 16 is a flowchart illustrating a manufacturing method of glenoid baseplate according to an embodiment of the present disclosure.

FIG. 17 is a flowchart illustrating an optimization step S20 according to an embodiment of the present disclosure.

FIG. 18 is a view illustrating a load acting on the scapula according to a movement of the shoulder joint.

FIG. 19 is a conceptual view illustrating an additive manufacturing step S50 according to an embodiment of the present disclosure.

### MODE FOR INVENTION

Hereinafter, embodiments of a glenoid baseplate of an artificial shoulder joint according to the present disclosure will be described in detail with reference to the accompanying drawings. It should be noted that like elements are indicated by like reference numerals throughout the drawings wherever possible. In addition, a detailed description of known functions and configurations incorporated herein may be omitted when it may obscure the subject matter of the present disclosure. Unless there is a special definition, all terms in this specification are the same as the general meaning of terms understood by those skilled in the art to which the present disclosure belongs, and when conflicting with the meaning of terms used in this specification, the terms used generally in the art follow the definition of the terms used herein. Throughout the specification, it will be understood that when a part is referred to as “including” an element, the part does not preclude other elements and may further include other elements unless stated otherwise. In addition, terms such as “part” and so on refer to units which perform at least one function or operation. In addition, when components are referred to as “connected”, it may mean that the components are not limited to being engaged in direct contact with each other, but includes being engaged through another component, and may be disposed such that a predetermined force or energy is capable of being transmitted even if the component is not engaged. Terms such as “first” and “second” may be used to indicate the same or substantially the same configuration in a different order, and may be interpreted as substantially the same configuration as a configuration that does not indicate “first”, “second”, and so on. Hereinafter, an exemplary embodiment of the present disclosure will be described with reference to the accompanying drawings to describe the present disclosure in detail.

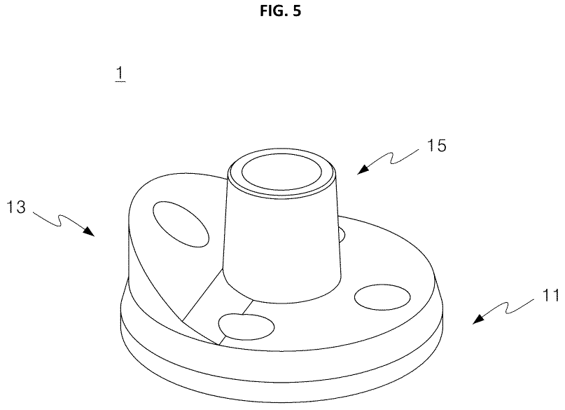

Referring to FIG. 5, a glenoid baseplate 1 according to the present disclosure forms an artificial shoulder joint together with other surgical apparatus such as a glenosphere, an insert, and so on. The glenoid baseplate 1 may accommodate a fixing means such as a screw, and the fixing means may engage with the bone or be inserted into the bone, so that the glenoid baseplate 1 is capable of being fixed to glenoid 911. The glenoid baseplate 1 may be configured to exhibit a required strength while being made lightweight. At least a portion of the glenoid baseplate 1 may have a solid surface. Here, a solid is a concept that contrasts with porosity. That is, the solid may be understood as a shape that is smooth, hard, or has no pores, unlike the porous layer 3 in which a plurality of pores is formed that will be described later. The glenoid baseplate 1 may include a base 11 settled on the glenoid 911, an augement 13 reinforcing the glenoid baseplate 1 at one side of the base 11, and a stem 15 that extends at a predetermined angle from one side of the base 11.

Referring to FIG. 5 to FIG. 8, the base 11 may be formed as a plate shape having a predetermined thickness. In an exemplary embodiment, the base 11 may have a circular plate shape. However, it does not exclude the base may have an atypical shape or an asymmetrical shape that is not a circular shape. It is preferable that the base 11 is formed as a solid object to exhibit the strength capable of withstanding an applied load and strength. The base 11 includes a lateral surface 11a facing laterally and a medial surface 11b facing medially relative to the center of the body, and is surrounded by a side surface 11c connecting the lateral surface 11a and the medial surface 11b to each other. Therefore, the base 11 may be a solid object disposed between the lateral surface 11a and the medial surface 11b. Although the lateral surface 11a and the medial surface 11b may be formed such that the lateral surface 11a and the medial surface 11b are substantially flat or not having curvatures, the lateral surface 11a and the medial surface 11b may be curved to have predetermined curvatures. Particularly, the medial surface 11b in contact with the bone may have a convex shape suitable for being fixed to the glenoid 911. As will be described later, the medial surface 11b as a first surface may form a contact surface with a surface of an augment 13. In an exemplary embodiment of the present disclosure, the lateral surface 11a and the medial surface 11b may be formed to be flat, and may extend to be perpendicular to a central axis X1 of a stem to be described later.

The base 11 may comprise an axis X2 penetrating the base vertically, or from lateral side to medial side. It is preferable that the axis X2 is substantially the same as an axis X1 of a stem that will be described later. In an embodiment of the present disclosure where a stem 15 is extended from a center of the base 11 described later, an axis X2 of the base 11 may be the same as a central axis X1 of the stem 15. In another embodiment of the present disclosure, when the stem 15 is spaced apart or offset from the center of the base 11, the axis X2 may be offset from the central axis X1 of stem at a predetermined distance.

In addition, the base 11 may comprise a central fixing hole 111, and a peripheral fixing hole 113 passing through the lateral surface 11a and the medial surface 11b as fixing holes.

The central fixing hole 111 is formed vertically pass through the base, or from lateral side to medial side. The central fixing hole 111 is formed in the center of the base 11, and a fixing means is inserted into for passing through the central fixing hole 111. Although the central fixing hole 111 is capable of being disposed in the center of base 11 and/or glenoid baseplate 1, even when the central fixing hole 111 is formed on a position that is deviated from the center, it can be understood as a concept that the central fixing hole 111 extends in a direction in which the lateral surface 11a and/or the stem 15 that will be described later are formed in contrast to the peripheral fixing hole 113. Since a stem 15 that will be described later may be extended having a hollow extending from the central fixing hole 111, a central axis of the central fixing hole and a central axis X1 of the stem 15 may substantially be the same axis.

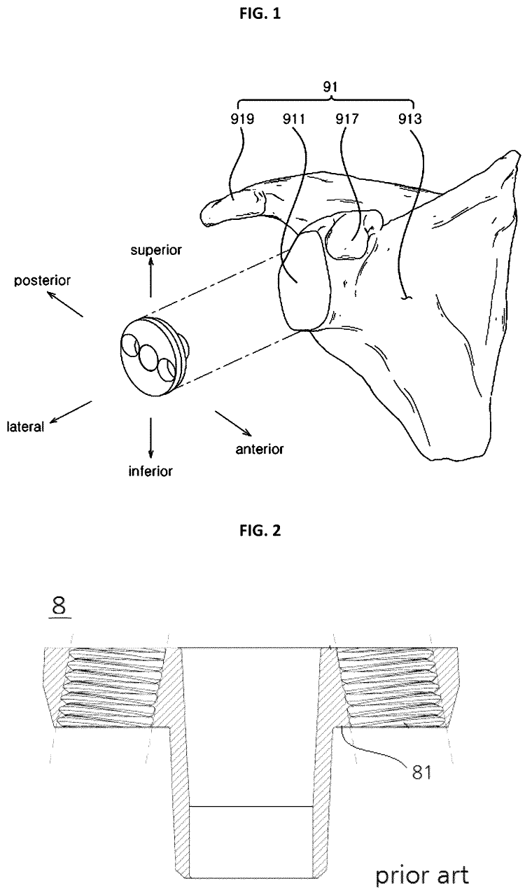

The peripheral fixing hole 113 is formed around the central fixing hole 111. In an embodiment of the present disclosure, total of four peripheral fixing holes 113 may be formed, one in each of four directions of the central fixing hole 111. However, this is only one embodiment of the present disclosure, and there is no limitation in the number and direction of the peripheral fixing holes 113 and/or a central angle between the centers of the peripheral fixing hole 113 with the central fixing hole 111 as a center. The peripheral fixing hole 113 may be defined as a hole defined by an inner circumferential surface that extends from a lateral surface 11a to a medial surface 11b, and may be formed in a tapered shape in which a width thereof becomes gradually reduced downward. In addition, the peripheral fixing hole 113 may be formed by forming a predetermined angle with a glenoid 911 (see FIG. 1) that is not perpendicular. This means that an opening of the peripheral fixing hole that is formed through the lateral surface 11a and the medial surface 11b may be an eccentric circle with eccentric center. In particular, when the peripheral fixing hole 113 is formed from top surface to the bottom or from the lateral surface 11a to the medial surface 11b, the peripheral fixing hole 113 may be formed in a direction that is inclined outward with respect to a center of the base. Here, the outward refers to a direction away from the center of the central fixing hole 111. A thread is formed on the inner circumferential surface of the peripheral fixing hole 113, and the fixing means having a corresponding thread may be fixed to the peripheral fixing hole 113.

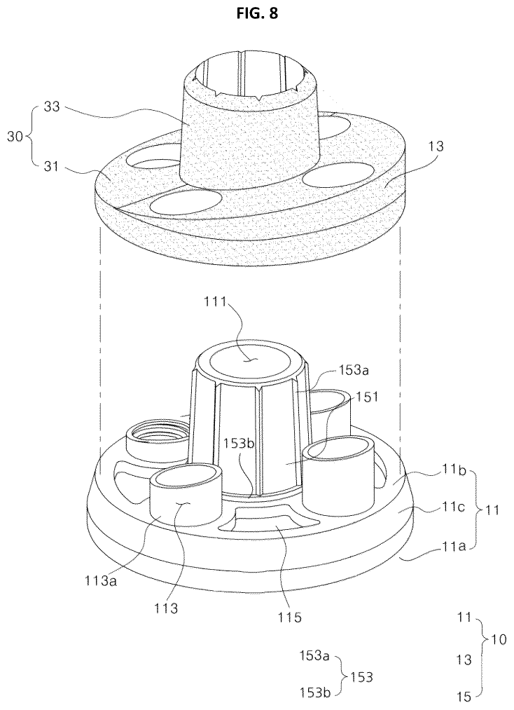

The base 11 may include a flange 113a that protrudes from a surface of the base 11 along an edge of the peripheral fixing hole 113. The flange 113a may be formed for exhibiting a proper strength of the glenoid baseplate. A larger stress may be generated around the central fixing hole 111 and/or the peripheral fixing hole 113 by the fixing means than in other portions of the base 11. The flange 113a may be provided such that the flange 113a forms the strength against such stress. Furthermore, the flange 113a may be provided such that the flange 113a is exposed toward a contact surface when the porous layer 3 that will be described later is coated and/or formed on the glenoid baseplate 1. When a fixing means is inserted into a bone region through the peripheral fixing hole 113, the flange 113a may guide the fixing means. The flange 113a may protrude from a surface of the base by a predetermined height. Referring to FIG. 8, the flange 113a may have different protruded height as the augment 13 is formed. However, it is preferable that the flange 113a is extended by a height corresponding to a length between the medial surface 11b and an augment 13 that will be described later. An extended surface of the flange 113a may be formed with a slope that has a first angle or a second angle with the medial surface 11b.

In another embodiment of the present disclosure, the base 11 may further include a recessed part 115. As illustrated in FIG. 8, the recessed part 115 is recessed at a surface of the base 11 with a predetermined depth. It is preferable that the recessed part 115 is formed on the medial surface 11b on which is formed in a direction facing the glenoid. The recessed part 115 may be formed by recessing a portion that is spaced at least a predetermined distance apart from at least one of the central fixing hole, the peripheral fixing hole, and an edge of the base. In an exemplary embodiment, the recessed part 115 may be formed in a portion spaced a predetermined length apart from the peripheral fixing hole 113 and is recessed while forming a boundary with the medial surface 11b. By recessing a portion where a relatively small stress is applied, a space in which a bone can grow may be maximized and sufficient strength may be exhibited. As such, the recessed part 115 may be formed such that the recessed part 115 is spaced a predetermined distance apart from the central fixing hole 111 and the edge of the base 11. The distance at which the boundary of the recessed part 115 is spaced apart from the peripheral fixing hole 113, the central fixing hole 111, and the edge of the base may vary according to a shape of the baseplate and the number of fixing holes. In another embodiment, the recessed part 115 may be formed at a predetermined depth in the lateral surface 11a or may be formed in both the lateral surface 11a and the medial surface 11b, thereby being capable of minimizing a thickness of a portion of the base 11 having a small influence on the strength exhibition of the baseplate. Several recessed parts 115 may be formed according to a shape of the base 11, the recessed part 115 may be recessed by a different depth for each portion, or a recessed depth in a recessed part 115 may be gradually changed.

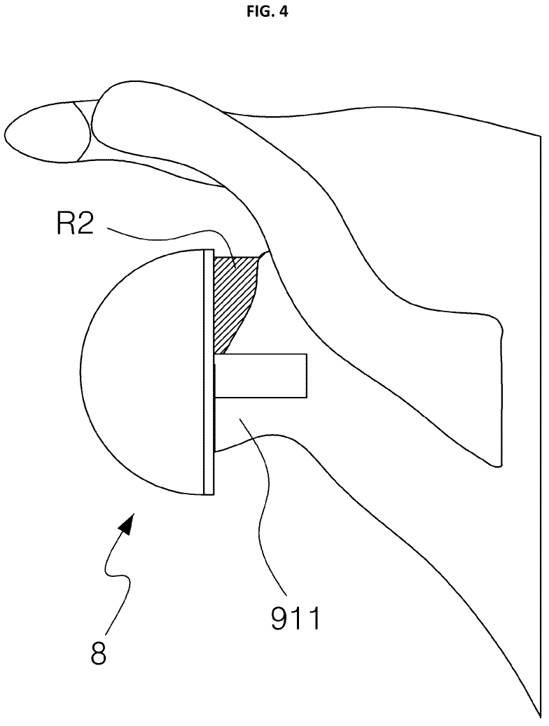

Referring to FIG. 5 to FIG. 7, the augment 13 may be disposed on the medial surface 11b, which is a first surface of the base 11, to compensate bone loss. As described above in FIG. 3 and FIG. 4, when a baseplate which cannot compensate bone loss is inserted for fixation at the scapular glenoid 911 that has suffered bone loss, there is a problem that the baseplate cannot be settled on the scapula 91 or glenoid 911 at a portion R2 where bone loss happened. Accordingly, the glenoid baseplate 1 of present disclosure is provided with an augment 13 for compensating bone loss and minimizing bone cutting. The augment 13 may be formed such that the augment 13 is spaced a predetermined distance apart from a central axis X1 of stem. Accordingly, the bone cutting would be reduced compared to the case where the augment 13 is formed from the central axis X1 of the stem.

A surface of the augment 13 makes a contact surface S with a first surface 11b, and the surface of the augment 13 may form a predetermined angle with the central axis X1 that is not perpendicular. In addition, the surface of the augment 13 may be formed to be inclined in a direction while having a first angle a1 with the first surface from a point a first distance D1 away from the axis X1. It is preferable that the first surface of the augment is extended to the opposite direction of a central axis from the first boundary 141 made by the first surface 11b and augment 13. More preferably, a direction of augment 13 extends while having the first angle al with respect to the first surface 11b may be defined as a direction of edge of the base opposite to the central axis X1 that is perpendicular to a first boundary 141 to be described later.

As will be described later, the augment 13 is formed in a porous structure having plural pores inside, as illustrated in FIG. 8, so that the augment 13 may induce bone growth between pores therein after procedure. In addition, the augment 13 may be formed integrally with the base 11. This comprises that the augment 13 of porous structure is formed integrally with the base 11. By forming the augment 13 and the base 11 integrally, the augment 13 and the base 11 do not peel off from each other, and member for binding components above may be omitted

The augment 13 comprises a first wedge 131 having a second surface 131a, a second wedge 133 having a third surface 133a. And a medial surface 11b, as a first surface, the second surface 131a and the third surface 133 form a contact surface S.

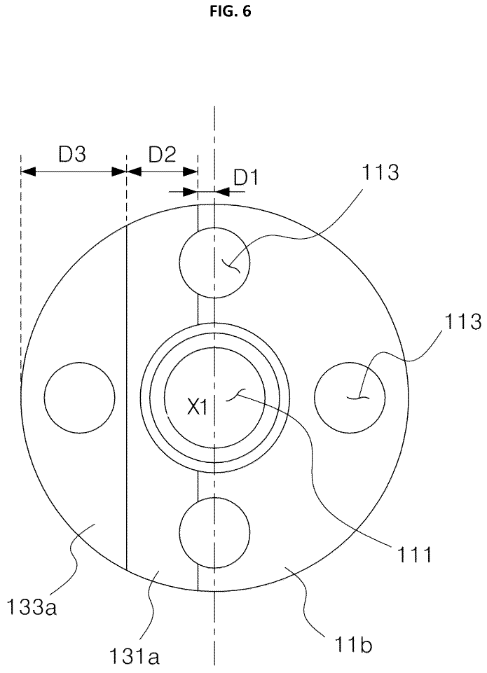

The first wedge 131 is extended portion from a region that is spaced a first distance D1 apart from the central axis of the augment 13, that is, the first boundary 141 that will be described later. The first wedge 131 has an extending second surface 131a while forming a first angle a1 with the medial surface 11b. The first wedge 131 extends from a region that is spaced a first distance D1 apart from a central axis X1, and may preferably extend from a region that is spaced a first distance D1 apart from a straight line or a chord passing through the central axis X1 on the base 11. It is preferable to form the first distance D1 to be shorter than the distance r from a central axis X1 to an outer surface 151 of the stem, which is the radius of the stem. The first wedge and a second wedge that will be described later enable stable mount of glenoid baseplate by compensating bone loss. In an exemplary embodiment of the present disclosure, the first wedge 131 extends in a direction of edge of the base 11, so that the second surface 131a forms a slope inclined in a direction, as shown in FIG. 6. The second surface 131a may extend to have a second distance D2 when the second surface 131a is projected onto the medial surface 11b.

The second wedge 133 is a portion that is provided on a side of a first wedge 131, and has a third surface 133a extending while forming a second angle a2 with the medial surface 11b. A second wedge 133 extends in a direction of an edge of the base 11, so that the third surface 133a forms a slope inclined in a direction, as shown in FIG. 6., and may extend to have a third distance D3 when the third surface 133a is projected onto the medial surface 11b. The third surface 133a bends and extends from the second surface, and a second angle a2 formed by the third surface 133a and medial surface 11b is different from a first angle a1. In an exemplary embodiment of the present disclosure, the second angle a2 may be larger than the first degree a1, and an augment having various angles such as 5°, 10°, 15°, etc. as the second angle may be disposed on the medial surface 11b. Accordingly, the glenoid baseplate may be settled on the glenoid in response to a more three-dimensional bone shape and/or a lost bone shape. And the bone cutting is reduced compared to the case where an augment has only one slope.

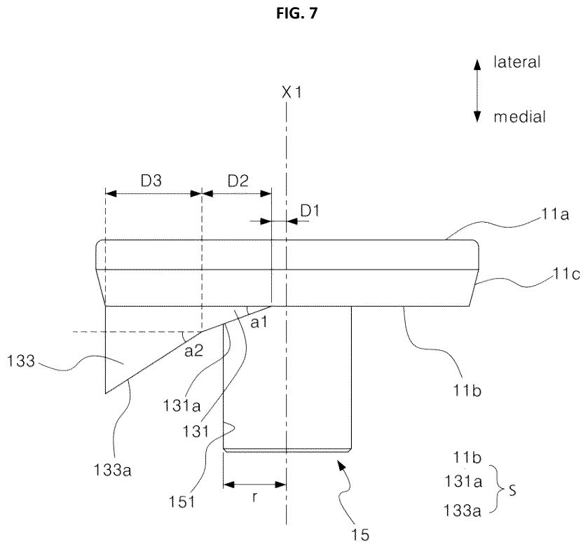

As illustrated in FIG. 6 and FIG. 7, in an embodiment of the present disclosure, the sum of a first distance D1, which is the distance from a central axis to a region where an augment extends, and a second distance D2, which is the distance that a second surface 131a extends in a direction horizontally or parallel to the medial surface 11b, may be formed to be larger than the distance r from the central axis X1 to an outer surface 151 of the stem. In addition, unlike what is illustrated in FIG. 6 and FIG. 7, in an embodiment of the present disclosure, the sum of the first length D1 and the second length D2 may be formed to be smaller than the distance r from central axis X1 to outer surface 151 of the stem. That is, the sum of distance D2, which is the distance of the second surface extended horizontally or parallel to the medial surface 11b and distance D1, which is the distance from the axis to the point where augment is extended, may be formed to be smaller than the radius of the stem.

Referring to FIG. 6 again, the augment 13 and the medial surface 11b, which is a first surface, may define a first boundary 141. And a second surface 131a of the first wedge 131 and a third surface 133a of the second wedge 133 may define a second boundary 143.

Since a second surface 131a makes an inclined slope to a direction, it is preferable that the first boundary 141 is formed in a straight line dividing a predetermined portion of the base 11. At this time, since the first wedge 131 is formed to be inclined in a direction from a region spaced a first length D1 apart from the axis X1, it is preferable that the first boundary 141 forms a straight line spaced a first length D1 apart from a straight line passing through the central axis X1 on the medial surface 11b. The first boundary 141 may extend from one end to the other end of the base 11, but a part of the first boundary 141 may be missed by the central fixing hole 111 and the peripheral fixing hole 113, as shown in FIG. 6.

A third surface 133a forms a slope inclined in a direction, so the second boundary 143 may be formed in a straight line dividing a predetermined portion of the base 11 on the same principle as the first boundary 141. It is preferable that the second boundary 143 is formed to be spaced a second length D2 apart from the first boundary 143. A part of the second boundary 143 may also be missed by the central fixing hole 111 and the peripheral fixing hole 113. In an embodiment of the present disclosure, the distance between central axis X1 and second boundary 143 may be formed to be smaller than the distance r between central axis X1 and outer surface of the stem. However, the distance between axis X1 and the second boundary 143 may also be greater than or equal to the distance r, which is the distance between the central axis X1 and outer surface of stem.

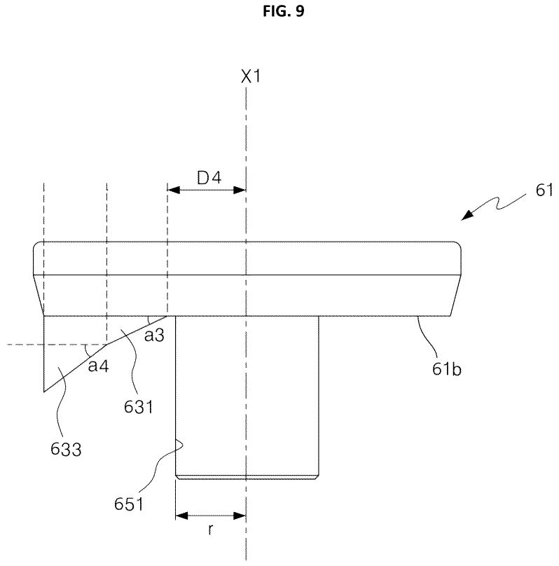

Referring to FIG. 9 and FIG. 10, augments 63, 73 according to other embodiments of the present disclosure will be described. In another embodiment of the present disclosure, in the augment 63, a medial surface 61b, a first surface of a first wedge 631 and a surface of a second wedge 633 form a contact surface, but the first wedge 631 may extend in a direction from the region spaced apart from the central axis by a fourth length D4, which is greater than the radius r of the stem. A third angle a3 formed by a first surface of a first wedge 631 with the medial surface 61b, and a fourth angle a4 formed by a surface of a second wedge 633 with the medial surface 61b may be formed variously, but the fourth angle a4 may be formed to be larger than the third angle a3, thereby minimizing the amount of bone cutting.

An augment 73 according to another embodiment of the present disclosure may be provided such that a surface contacting a valley of the first wedge 731 and second wedge 733 may form a curved surface. Each curved surfaces of the first wedge 731 and second wedge 733 may extend in a direction in which arcs having a first radius C1 and a second radius C2 extends to a direction of the first boundary 741 is extended. At this time, the first radius C1 may be formed to be larger than the second radius C2, so that a surface of the first wedge 731 extends with smaller curvature than a curvature of a surface of the second wedge 733.

Referring to FIG. 5 to FIG. 7 again, the stem 15 has a central axis X1 from the medial surface 11b, which is a first surface of the base 11, and extends unilaterally in a direction. The stem 15 is preferably cylindrical, and is inserted into the glenoid 911 of the scapula 91. At this time, in order to secure an optimal fixing force between the scapula 91 and the glenoid baseplate 1, it is preferable that an axis of stem 15 is vertically deployed to the glenoid 911. The stem 15 forms a hollow which is in communication with the central fixing hole 111 and which is a channel where fixing means passes therethrough. The stem 15 may have a cylindrical column shape having a predetermined inner diameter. However, in some embodiments, the stem 15 may be formed to be tapered as the stem 15 extends from the base to an end of the upper side. Accordingly, the stem 15 may have an outer surface 151 having a predetermined radius from the central axis X1 or being tapered. The stem 15 may form an opening smaller than a hollow formed at an extended end, so that fixing means passing through the central fixing hole 111 is caught to specify a position. In addition, an edge of the stem 15 is chamfered, so that the stem 15 is capable of being easily inserted into the glenoid 911 of scapula 91. It is preferable that the stem 15 extends perpendicularly from the base 11, so that the central axis X1 of the stem 15 is perpendicular to the base 11 and the medial surface 11b, which is a first surface. In addition, it is preferable that the stem 15 is formed from center of the base 11, so that the axis X2 of base 11 or baseplate 1 is the same as the central axis X1 of the stem 15.

Referring to FIG. 8, the stem 15 may comprise a reinforcement member 153 that protrudes radially outward from an outer surface 151. The reinforcement member 153 may be provided such that the rigidity and/or the strength of the stem 15 are reinforced. The reinforcement member 153 may be formed of plural ribs 153a having a predetermined width and extending in a longitudinal direction of the stem 15. In an exemplary embodiment of the present disclosure, eight ribs 153a may be formed at a central angle of 45 degrees from the central axis X1 of the stem 15. However, it is possible that four, twelve, or ten ribs 153a may be formed, and the central angle of the plural ribs 153a may not be constant and may be different from each other. Particularly, the rib 153a may be formed to be concentrated on a predetermined portion of the stem according to a movement shape of the shoulder joint and/or the shape of the baseplate. In addition, the rib 153a may extend with a spiral shape on the stem. Furthermore, the reinforcement member 153 may be formed of a ring shaped rim 153b with a predetermined width on at least one end portion of the stem and be protruded from the stem. The reinforcement member 153 as the rim 153b may be formed in an annular shape from a base-side end of stem 15, or may be formed in an annular shape from an end in a direction in which the reinforcement member 153 is inserted into the glenoid, or may be formed in an annular shape from both ends of the stem 15. In an exemplary embodiment of the present disclosure, the reinforcement member 153 may simultaneously comprise a rib 153a and a rim 153b protruding from the outer surface 151 of the stem, respectively.

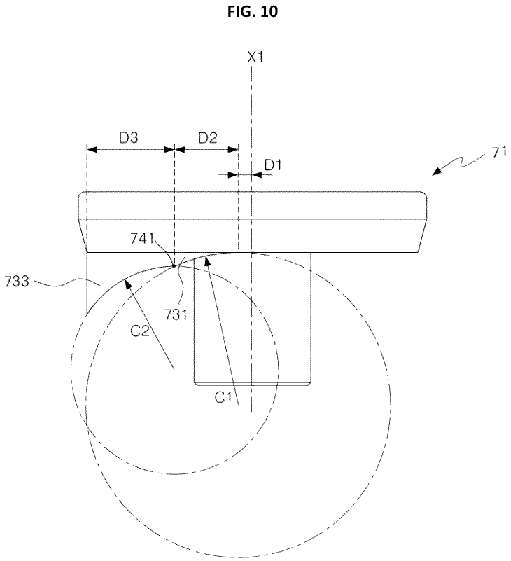

Referring to another embodiment of the present disclosure illustrated in FIG. 11, the stem 15 may be formed to be offset by a predetermined distance from center of the baseplate 1. As described in FIG. 11, the stem 15 may be formed to be offset in a direction in which the augment 13 is not formed, but may be formed to be offset in a direction in which the augment 13 is formed. At this time, the augment 13 may be integrally formed with the base 11 at a portion spaced a first length D1 apart from the central axis X1 of the stem, and a first boundary 141 between a first surface 11b and the augment 13 may be formed in a straight line spaced a first length D1 apart from the central axis X1 of the stem.

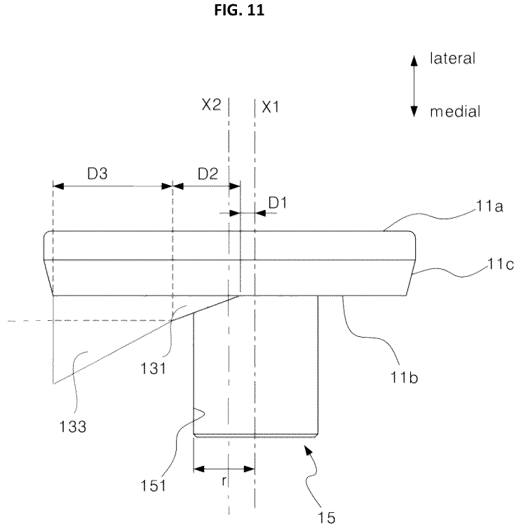

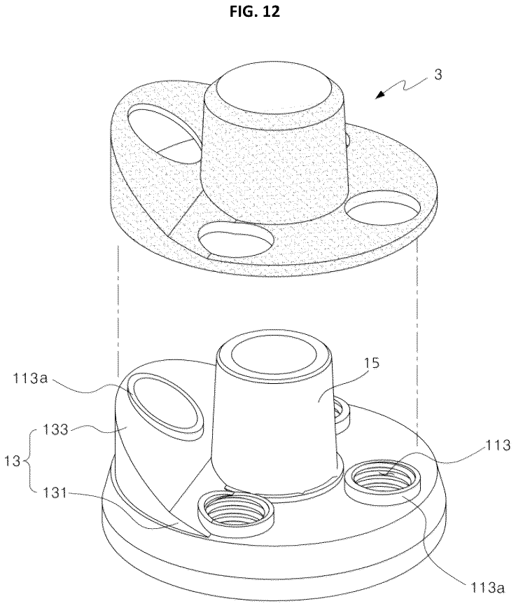

Referring to the FIG. 8, FIG. 12 and FIG. 13, a glenoid baseplate may comprise a porous layer 3, or as described above, the augment 13 may be formed in a porous structure. The porous layer 3 is formed to have a predetermined thickness on a side of the base 11 and the stem 15, while coating the surface of the base 11 and stem 15, and has a three-dimensional structure forming a pore therein, without any particular constraints on the shape of the pore. The porous layer 3 may induce bone growth between internal pores to promote fusion between bone defects and bone fractures, or to achieve post-operative strength that is difficult to achieve with an artificial joint alone. The material constituting the porous layer 3 is not limited to any specific concept, but may preferably be titanium. The porous layer 3 may be formed through a 3D printing method and so on, by using titanium powder, or alloy powder based on titanium. After the 3D printing and so on are performed, the porous layer 3 is completed by performing a post-process such as cleaning and so on. That is, the porous layer 3 forms pores on outer surface of the lateral surface 11a of the base and the outer surface of stem 15 by using a biocompatible material powder such as titanium powder, titanium alloy powder, cobalt chromium powder, thereby being capable of increasing a coupling force with the bone using bone ingrowth into the pores when the porous layer 3 is implanted into the human body. The porous layer 3 and the solid surface of glenoid baseplate 1 may be formed with the same material.

As shown in FIG. 12, in another embodiment of the present disclosure, when the augment 13 has a solid surface, the porous layer 3 is provided on the augment 13 with a predetermined thickness, thereby inducing bone growth between internal pores.

The porous layer 3 has a complementary shape to the base 11 and the stem 15, and comprises a first layer 31 formed corresponding to the base 11 and a second layer 33 formed corresponding to the stem 15.

Referring to FIG. 13, the first layer 31 is a part formed on a surface of the base 11, preferably, on a medial surface 11b, with a predetermined thickness. The first layer 31 comprises a through hole 311 corresponding to the central fixing hole 111 and the peripheral fixing hole 113 and a protrusion 313. In another embodiment, the first layer 31 may be formed up to the lateral surface 11c of the base to coat the circumference of the base 11. As shown in FIG. 8, when the augment 13 is formed in a porous structure, the augment 13 may be provided at the first layer 31.

The through-hole 311 is a portion in which an opening is formed in response to the peripheral fixing hole 113 formed to allow fixing means to pass through. The through-hole 311 may be formed to be larger than the peripheral fixing hole 113 by a predetermined amount, so that the flange 113a is capable of passing through the through-hole 311 and is capable of being exposed toward a direction toward the glenoid.

The protruded part 313 is formed corresponding to the recessed part 115 formed on the medial surface 11b of the base described above, and may protrude by a height at which the recessed part 115 is recessed from the medial surface 11b such that the protrusion part 313 has a complementary shape with the medial surface 11b. Accordingly, pores are increased, enabling more bone growth.

The second layer 33 is a portion formed by a predetermined thickness while surrounding the surface of the stem 15 and the outer surface of the reinforcing member 153. And the second layer 33 comprises a recessed line 331 formed corresponding to a rib 135a and a rim 135b. It is preferable that the recessed line 331 is formed by being recessed by the protruding width and the protruding thickness of the rib 135a and the rim 135b.

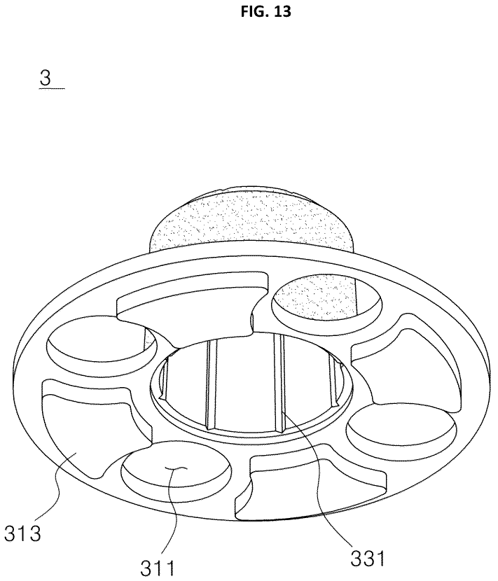

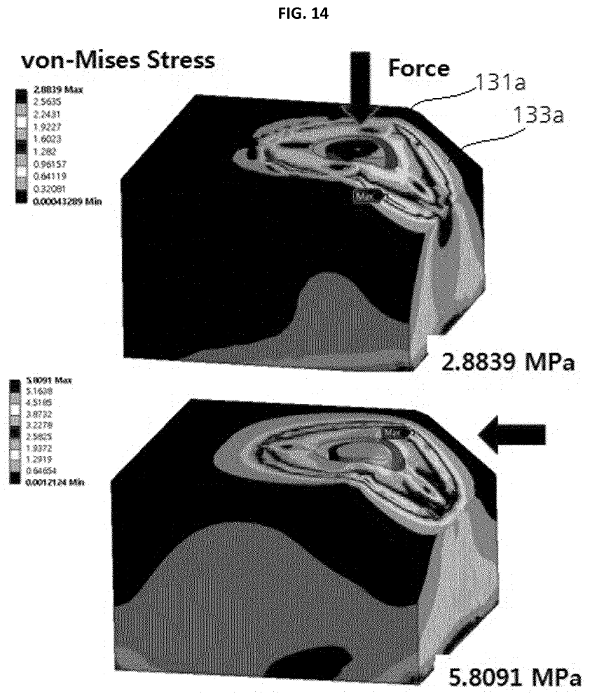

Referring to the FIG. 14, and FIG. 15, a case where both the second surface 131a and the third surface 133a according to an embodiment of the present disclosure will be compared with a case where an inclined surface is formed on the augment. FIG. 14 and FIG. 15 illustrate stress generated in a valley part according to a direction in which an external force is applied when the glenoid baseplate is settled. In an embodiment in which a second surface 131a and a third surfaces 133a of the augment 13 are formed as shown in FIG. 14, the valley receives relatively little stress against external forces applied in all directions, whereas in another embodiment in which the second surface 131a is missing and only one slope is provided as shown in FIG. 15, the valley receives relatively large stress against external forces applied in all directions. Therefore, as in this present disclosure, both the second surface 131a and the third surface 133a must be provided to improve the durability of the glenoid baseplate, promote recovery of the surgical site after surgery, and reduce the probability of aftereffects.

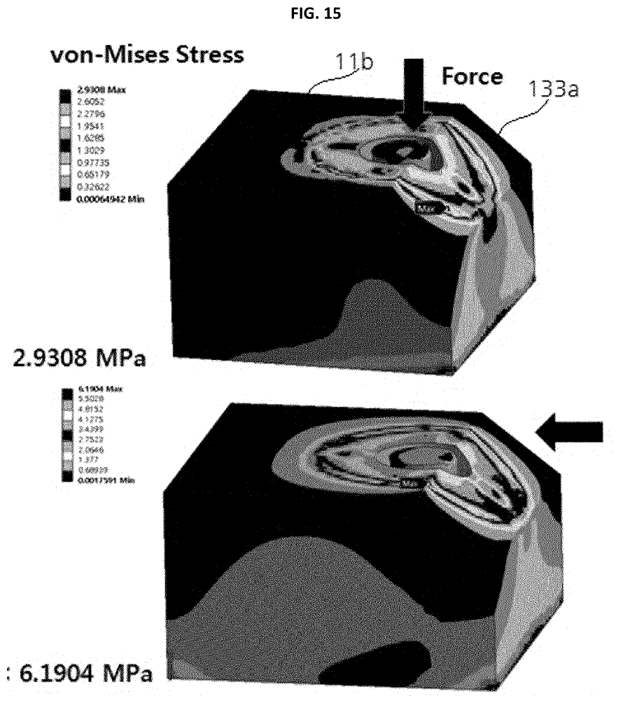

Hereafter, referring to FIG. 16 to FIG. 19, a manufacturing method of glenoid baseplate S1 according to an embodiment of the present disclosure will be described. The manufacturing method S1 of glenoid baseplate allows the baseplate inserted and/or settled in the patient's glenoid to express the necessary strength while being lightweight. And the manufacturing method S1 forms a part facing the bone recessed, and maximizes the formation of porous layer, so that the bone growth is maximized after the glenoid baseplate is settled, and rapid recovery after surgery can be expected. The manufacturing method S1 of glenoid baseplate comprises a shape determination step S10, an optimization step S20, a detailed design step S30, and an additive manufacturing step S50.

The shape determination step S10 is a process of determining a shape of glenoid baseplate, and is a process of determining size and shape such as the overall shape of the glenoid baseplate 1, size, position and number of central fixing holes 111 and peripheral fixing holes 113, extension length of the stem 15, and so on. Furthermore, in the shape determination step S10, shape of the augment 13 described above, angle formed by the second surface and third surface with the first surface, extended length, et cetera may be determined.

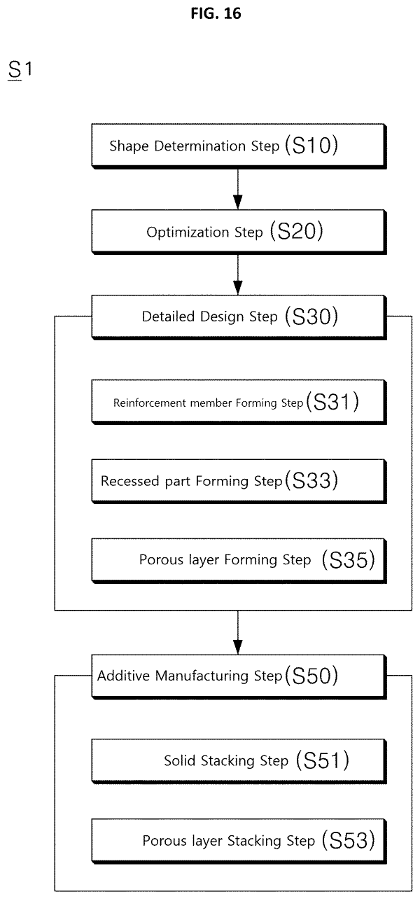

Referring to FIG. 17, the optimization step S20 is a process of deriving the optimal shape of the glenoid baseplate based on a load and a constraint condition acting on the glenoid baseplate. In an embodiment, the optimal shape may be derived by topology optimization design method. The topology optimization design is a structural optimization design method that optimizes the connectivity of each element constituting the structure to achieve an objective function while satisfying a design condition. The topology optimization design may solve a problem in which a phase occurring during the shape optimization step is fixed, and has an advantage of increasing the degree of freedom. Therefore, in the glenoid baseplate manufacturing method S1 according to an embodiment of this present disclosure, the optimal shape of baseplate may be derived based on an input value and a constraint condition. At this time, an optimal shape where the position of the central fixing hole 111, the position of the peripheral fixing hole 113, the length of the stem 15, and so on are determined in the shape determination step S10, may minimize the material used in the portion with less stress and deformation for weight lightening and more bone growth. The optimization step S20 may include a region setting step S21, a constraint setting step S23, a load determination step S25, and a calculation step S27, and the optimization step S20 may be performed multiple times.

The region setting step S21 is a process of setting a region to be optimized, through analysis. In the region setting step S21, a region to be optimized is designated so that the required strength is exhibited and the glenoid baseplate is made lightweight, so that a portion accommodating the fixing means or a portion in contact with bone or tissue may be excluded from the region to be optimized. In an exemplary embodiment of the present disclosure, the central fixing hole 111, the peripheral fixing hole 113, and the flange 113a may be set as non-designed region to prevent unnecessary degradation of strength. In particular, in the region setting step S21, the glenoid baseplate 1 may be divided into several parts, and optimization for other parts may be performed after optimization is performed on one part.

The constraint setting step S23 is a process of setting an optimization constraint together with the objective function, which is the goal of optimization, while the objective function expresses rigidity, and the constraint may set a volume and weight equal to or less than a predetermined degree for weight reduction. In one embodiment, the objective function of the glenoid baseplate may be set to express rigidity capable of withstanding the applied load, and the constraint condition may set a volume of 80% or less.

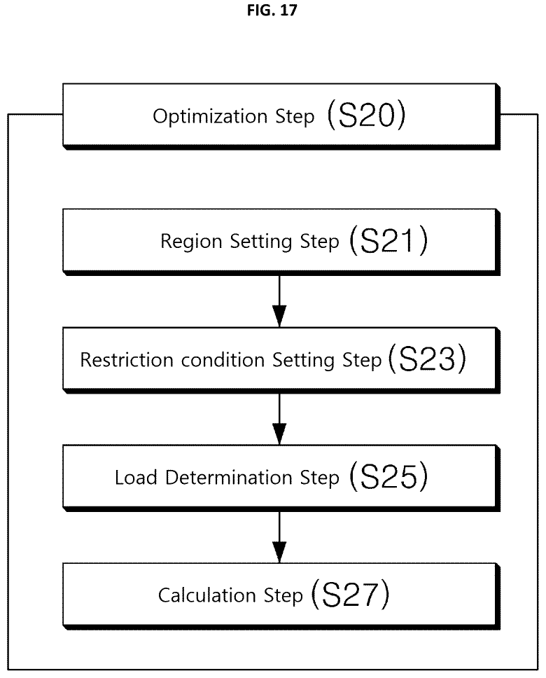

The load determination step S25 is a process of setting a load and a constraint condition applied to the glenoid baseplate. The load applied to the glenoid baseplate may be considered by individually or overlapping, depending on the movement of the shoulder joint. The load may be considered as concentrated load, pressure, forced displacement, and so on. As shown in FIG. 18, in one embodiment, the scapula rotates about 30° from 90° abduction to form a 60° scapulohumeral angle, and the maximum scapulohumeral joint force occurs near the 90° abduction of the inverted prosthesis in the absence of the supraspinatus muscle, so a force may act obliquely about 30° from vertical to the base. In addition, compressive force, moment, and so on may occur depending on the movement of the arm. In addition, constraints on the glenoid baseplate are set, which may be surface constraint or hinge constraint of a predetermined portion of the baseplate. In an embodiment of the present disclosure, the base portion may be surface-constrained and interpreted.

The calculation step S27 is a process of deriving an optimal shape through analysis, which may deduce the optimal shape of the lightweight glenoid baseplate expressing an appropriate strength. By the calculation step S27, the thickness or width of the base 11, the augment 13, and the stem 15 may be optimized, and the recessed portion 115 and the reinforcement member 153 may be formed, as described above. In particular, the calculation step S27 may have substantially the same configuration as the detailed design step S30 described later. In some embodiments, both the dimensions and cross-sectional shapes of the recessed part 115 and the reinforcing member 153 may be determined in the calculation step S27, but for convenience of processing and additive manufacturing, this may be additionally determined or considered in the detailed design step S30.

The detailed design step S30 is a process of determining the detailed shapes of the glenoid baseplate according to the optimal shape determined by the optimization step, and in one embodiment, the dimensions of the recessed part 115, the stem 15, and the reinforcement member 153 and the cross-sectional shape and the thickness of the porous layer may be determined. The detailed design step S30 may comprise a reinforcement member forming step S31, a recessed part forming step S33, and a porous layer forming step S35.

The reinforcement member forming step S31 is a process of determining a reinforcement member formed around the stem of the glenoid baseplate. It is possible to reduce the weight while securing the rigidity of the stem 15 by determining at least one of the cross-sectional shape, number, extension length, and center angle of a rib protruding from the outer surface 151 of the stem with a predetermined width and a rim protruding from the outer surface 151 of the stem in an annular shape with a predetermined width at at least one end of the stem.

The recessed part forming step S33 is a process of determining a recessed part formed from the base, and at least one of a surface recessed from the base, a recessed depth, and a spacing distance may be determined.

The porous layer forming step S35 is a process of determining the pores and thickness of a porous layer formed to coat the surface of a solid region comprising a base and a stem, and the porous layer 30 is formed complementary to the upper surface 11a of the base and the outer surface of the stem 15 to maximize bone growth.

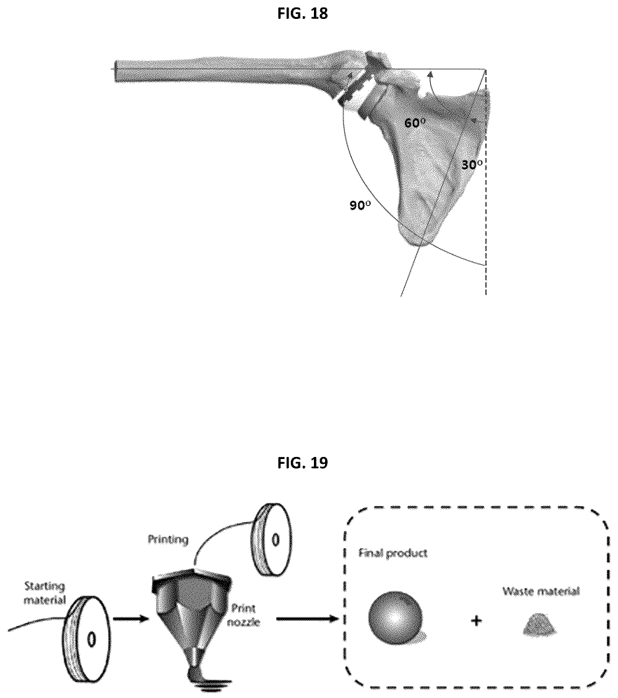

Referring to FIG. 19, the additive manufacturing step S50 is a process of manufacturing the determined solid region and porous layer through an additive method, and 3D printing may be performed by supplying metal powder to the implant surface along with an auxiliary gas, melting it with a laser heat source, and stacking the metal to a thickness of several mm or more. When glenoid baseplate is additively manufactured, the porosity and pore size can be stacked to an optimal size that induces bones to grow well inside the structure, so intra-bone growth can be achieved well, thereby increasing the initial fixing force. The additive manufacturing step S50 comprises a solid stacking step S51 and a porous layer additive step S53.

The solid stacking step S51 is a process of additively manufacturing a solid region comprising a base 11 and a stem 15 determined through the process up to the detailed design step S30. The solid stacking step S51 is substantially a kind of additive manufacturing and may preferably be performed using a 3D printer.

The porous layer stacking step S53 is a process of coating a solid region with a porous layer. The porous layer laminated in the porous layer stacking step S53 preferably has a complementary shape to one surface of the solid region comprising a base and a stem, and the porous layer is coated on the solid region while forming plural pores, so that the density of the porous layer is lower than that of the solid region, enabling the baseplate to be lightweight, and the relative porosity is further secured to improve bone growth. In addition, the porous layer and the solid region may be integrally stacked in the porous layer stacking step S53.

Preferably, the solid region stacked in the solid stacking step S51 and the porous layer stacked in the porous layer stacking step S53 are desirably stacked with the same material.

The foregoing detailed description is for illustrative purposes only. In addition, the description provides an exemplary embodiment of the present disclosure, and the present disclosure may be used in other various combination, changes, and environments. That is, the present disclosure may be changed or modified within the scope of the present disclosure described herein, a range equivalent to the description, and/or within the knowledge or technology in the related art. The embodiments show an optimum state for achieving the spirit of the present disclosure, and various modification required for specific applications and uses of the present disclosure are also possible. Therefore, the detailed description of the present disclosure is not intended to limit the present disclosure in the embodiment. In addition, the claims should be construed as including other embodiments.

1. A glenoid baseplate comprising:
a base configured to be settled on a scapular glenoid and having a first surface;
an augment disposed on the first surface; and
a stem extending from the first surface with a central axis,
wherein the first surface and one surface of the augment make a contact surface, while at least a part of the contact surface makes a predetermined angle, which is not perpendicular to the central axis of the stem.

2. The glenoid baseplate of claim 1, wherein the augment is formed to be spaced apart from the central axis,
and one surface of the augment is formed to be inclined while having a first angle with the first surface in one direction, from a point spaced apart from the central axis by a first length.

3. The glenoid baseplate of claim 2, wherein a first boundary between the augment and the first surface is formed as a straight line spaced a first length apart by from a straight line passing through the central axis on the first surface.

4. The glenoid baseplate of claim 3, wherein the augment comprises a first wedge having a second surface extending from the first boundary and a second wedge having a third surface bending and extending from the second surface.

5. The glenoid baseplate of claim 4, wherein a second angle formed by the third surface and the first surface is greater than the first angle formed by the second surface and the first surface.

6. The glenoid baseplate of claim 5, wherein a second boundary formed by the second surface and the third surface is formed as a straight line that is spaced a second length apart by the first boundary.

7. The glenoid baseplate of claim 3, wherein the first length is less than the distance between the central axis and an outer surface of the stem.

8. The glenoid baseplate of claim 7, wherein a distance between the central axis and the second boundary is less than the distance between the central axis and the outer surface of the stem.

9. The glenoid baseplate of claim 1, the glenoid baseplate further comprising a porous layer having a predetermined thickness on one side of the base and the stem, and having plural pores therein, wherein the porous layer has a complementary shape to the base and the stem

10. The glenoid baseplate of claim 9, wherein the base comprises:
a central fixing hole formed to vertically pass through the base and the stem;
a peripheral fixing hole formed around the central fixing hole; and
a flange protruding from a surface of the base along the edge of the peripheral fixing hole;
wherein one end of the stem and an edge of fixing hole vertically penetrating the base are exposed without being covered by the porous layer.

11. The glenoid baseplate of claim 9, wherein the augment is formed as a porous structure having plural pores therein.

12. The glenoid baseplate comprising:
a base configured to be settled on a scapular glenoid and having a first surface;
an augment disposed on the first surface; and
a stem extending from the first surface with a central axis,
wherein the augment comprises:
a first wedge having a second surface extending to one side from a point spaced a first length apart from the central axis and forming a first boundary with the first surface;
a second wedge having a third surface extending from the first wedge, and
at least one of the second surface and the third surface has an arc with predetermined curvature extending in a direction in which the first boundary is extended.

13. The glenoid baseplate of claim 1, wherein the stem comprises a reinforcement member extending along the outer surface; and the reinforcement member comprises a rib protruding and extending from one end of the stem to the other end of the stem with a predetermined width.

14. The glenoid baseplate of claim 13, wherein the reinforcement member further comprises a rim protruding in a ring shape with a predetermined width from at least one end of the stem.

15. The glenoid baseplate of claim 1, wherein the base comprises a central fixing hole formed vertically through the base, a peripheral fixing hole formed around the central fixing hole, and a recessed part recessed and formed by a predetermined depth in at least one surface of the base,
while the recessed part is recessed on a surface of the base at least a predetermined distance from the edge of at least one of the central fixing hole, the peripheral fixing hole, and the edge of the base.
